(19) United States
(12) Patent Application Publication
(10) Pub. No.: US 2024/0057860 A1
(43) Pub. Date: Feb. 22, 2024

(54) MEDICAL DEVICES AND RELATED METHODS THEREOF

(71) Applicant: Boston Scientific Scimed, Inc., Maple Grove, MN (US)

(72) Inventors: Juntong CHEN, Boston, MA (US); Oliver J. GORST, Lowell, MA (US); Jeffrey V. BEAN, Fitchburg, MA (US)

(73) Assignee: Boston Scientific Scimed, Inc., Maple Grove, MN (US)

(21) Appl. No.: 18/451,468

(22) Filed: Aug. 17, 2023

Related U.S. Application Data
(60) Provisional application No. 63/371,916, filed on Aug. 19, 2022.

(51) Int. Cl. A61B 1/32; A61B 1/06

(52) U.S. Cl. CPC A61B 1/32; A61B 1/0676

(57) ABSTRACT

A medical device comprises a handle including an actuator, a shaft extending distally from the handle, wherein the shaft includes a lumen, and a distal cap. The distal cap includes a body including a first channel, wherein the first channel is in communication with a distal portion of the lumen, a distal face including an opening, wherein the opening is a distal opening of the channel, and an expandable portion coupled to the body, wherein the expandable portion is configured to expand radially outwards from the body and to retract radially inwards towards the body from an expanded position. The expandable portion includes at least one light.

### CROSS-REFERENCE TO RELATED APPLICATIONS

This application claims the benefit of priority of U.S. Provisional Patent Application No. 63/371,916, filed Aug. 19, 2022, the entirety of which is incorporated herein by reference.

### TECHNICAL FIELD

This disclosure relates generally to medical devices and related methods. More particularly, at least some embodiments of the disclosure relate to medical devices, e.g., scopes, including expandable distal portions that allow for larger working channels and/or enhanced lighting, and related methods.

### BACKGROUND

Medical scopes often used in endoscopic procedures, e.g., duodenoscopes, catheters, etc., typically include accessory/working channel(s), irrigation channel(s), suction channel(s), lights, and/or an imager. For various reasons, an outer diameter of such scopes may be constrained to a certain dimension, e.g., 3.5 mm, thereby limiting a surface area of the distal face of the scope. Thus, in order to accommodate the various features on the distal face of the scope, the diameter or width of such features, e.g., a working channel diameter, the size of the lights, etc., is also necessarily limited. As a result, when operating these medical scopes, physicians may be limited to, for example, certain accessories and/or tools that are small enough to extend through the working channel. Similarly, physicians may be limited to, for example, lights or illumination devices that are smaller and/or of lesser capacity than lights or illumination devices that are larger.

### SUMMARY OF THE DISCLOSURE

Examples of the present disclosure relate to, among other things, medical devices and methods. Each of the examples disclosed herein may include one or more of the features described in connection with any of the other disclosed examples.

According to one aspect, a medical device may comprise a handle including an actuator, a shaft extending distally from the handle, wherein the shaft includes a lumen; and a distal cap, wherein the distal cap includes a body including a first channel, wherein the first channel is in communication with a distal portion of the lumen, a distal face including an opening, wherein the opening is a distal opening of the channel, and an expandable portion coupled to the body, wherein the expandable portion is configured to expand radially outwards from the body and to retract radially inwards towards the body from an expanded position, and wherein the expandable portion includes at least one light.

According to another aspect, the actuator may be in engagement with the expandable portion so that the actuator is configured to control the expansion and the retraction of the expandable portion.

According to another aspect, the expandable portion may be biased towards the retracted position. In the retracted position, an outer diameter of the distal cap may be approximately the same as an outer diameter of a remaining portion of the shaft.

According to another aspect, the distal face may not include any lights.

According to another aspect, a diameter of the opening of the distal face may be about 1.5 mm to 2.0 mm.

According to another aspect, the expandable portion may be coupled to the body via a hinge. The expandable portion may be coupled to a distal end of the body so that the expandable portion covers at least a portion of the opening in the retracted position and exposes an entirety of the opening in the expanded position. The expandable portion may be semi-annular so that in the retracted position a portion of the opening remains exposed. The expandable portion may be transparent. The actuator and the expandable portion may engage one another via an opening wire and a closing wire, wherein a force applied to the opening wire is configured to expand the expandable portion, and wherein a force applied to the closing wire is configured to retract the expandable portion. The body may further include an outer cylinder and an inner cylinder, wherein the outer cylinder encloses at least a portion of the inner cylinder and the inner cylinder may be rotatable about a central longitudinal axis of the body. The outer cylinder may include a first slot and the inner cylinder may include a second slot, wherein the first slot and the second slot are overlaid and the first slot and second slot are configured to receive the expandable portion in the retracted position. The expandable portion may be coupled to the outer cylinder via a hinge and the inner cylinder via a hinge, and a rotational force applied to the rotatable inner cylinder in a first direction may be configured to expand the expandable portion. A rotational force applied to the rotatable inner cylinder in a second direction may be configured to retract the expandable portion into the first slot and the second slot.

According to another aspect, a medical device end cap may comprise a body including a channel, a distal face including an opening, wherein the opening is a distal opening of the channel, a first wing coupled to the body, and a second wing coupled to the body, wherein each of the first wing and the second wing is configured to expand radially outwards from the body to an expanded position and to retract radially inwards towards the body from the expanded position, and wherein each of the first wing and the second wing includes at least one light.

According to another aspect, the first wing may be configured to expand radially outwards in a first radial direction and the second wing may be configured to expand radially outwards in a second radial direction, wherein the first radial direction and the second radial direction are different.

According to another aspect, the distal face may not include any lights.

According to another aspect, one or more of the first wing and the second wing may further include an imaging device.

In another aspect, a method of using a medical device, the medical device including a handle including an actuator, a shaft, and a distal cap including an expandable portion controllable by the actuator, wherein the expandable portion includes at least one light, may comprise delivering a distal end of the shaft and the distal cap into a bodily lumen, positioning a distal end of the shaft and the distal cap adjacent to a target site, actuating the actuator to control the expandable portion of the distal cap to expand the expandable portion of the distal cap, and activating the at least one light.

### BRIEF DESCRIPTION OF THE DRAWINGS

The accompanying drawings, which are incorporated in and constitute a part of this specification, illustrate various exemplary embodiments and together with the description, serve to explain the principles of the disclosed embodiments.

FIG. 1 is a perspective view of a medical device, according to an embodiment.

FIGS. 2A and 2B are perspective views of a distal aspect of the medical device according to FIG. 1 in different configurations.

FIGS. 3A-3C are perspective views of a distal aspect of a medical device, according to another embodiment.

### DETAILED DESCRIPTION

Reference will now be made in detail to aspects of the present disclosure, examples of which are illustrated in the accompanying drawings. Wherever possible, the same or similar reference numbers will be used through the drawings to refer to the same or like parts. The term “distal” refers to a portion farthest away from a user when introducing a device into a subject (e.g., patient). By contrast, the term “proximal” refers to a portion closest to the user when placing the device into the subject.

Both the foregoing general description and the following detailed description are exemplary and explanatory only and are not restrictive of the features, as claimed. As used herein, the terms “comprises,” “comprising,” “having,” “including,” or other variations thereof, are intended to cover a non-exclusive inclusion such that a process, method, article, or apparatus that comprises a list of elements does not include only those elements, but may include other elements not expressly listed or inherent to such a process, method, article, or apparatus. In this disclosure, relative terms, such as, for example, “about,” “substantially,” “generally,” and “approximately” are used to indicate a possible variation of ±10% in a stated value or characteristic.

Embodiments of the disclosure may solve one or more of the limitations in the art. The scope of the disclosure, however, is defined by the attached claims and not the ability to solve a specific problem. The disclosure, in certain embodiments, is drawn to a medical device, which may be, as an example, any scope (e.g., bronchoscope, duodenoscope, endoscope, colonoscope, ureteroscope, cholangioscope, etc.), including a handle, and a shaft, e.g., a catheter, that extends distally from the handle. As further discussed below, the shaft may include a distal cap including one or more “expandable” features, e.g., features configured to open/close, flare radially outwards, retract radially inwards, etc. in some embodiments, the expandable features do not increase the cross-sectional diameter (or otherwise affect the size) of the distal cap or distal portion of the shaft until expanded, so that the shaft may be of the same diameter during insertion/delivery. Each expandable feature may include a light, an imager, and/or additional scope features. Such a distal cap adds surface area to the distal face of the scope, relative to scopes including the light, the imager, and additional features on the distal face of the scope.

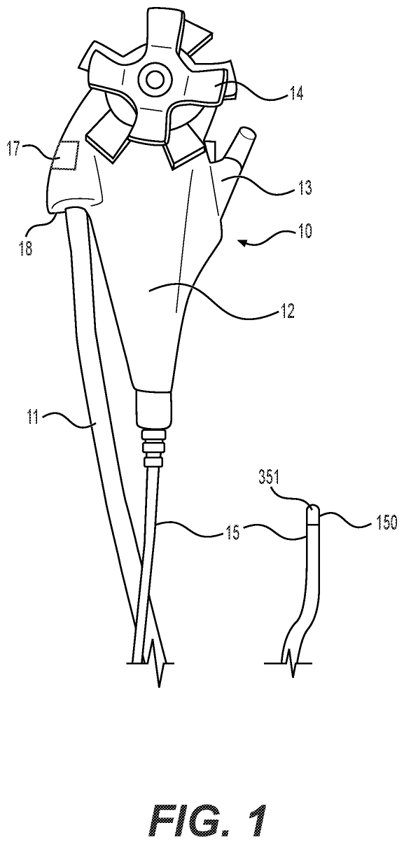

FIG. 1 shows a medical device 10 (device 10), e.g., a scope, according to an embodiment. Device 10 includes a flexible shaft 15 (e.g., a catheter) coupled to a distal cap 150, and a handle 12 connected to a proximal end of shaft 15. As discussed below, distal cap 150 may include one or more expandable/retractable features.

Handle 12 may be coupled to an umbilicus 11 and handle 12 may include an actuator 14, e.g., one or more knobs. Umbilicus 11 extends proximally relative to handle 12. Umbilicus 11 may be coupled to any suitable source, e.g., fluid, suction, electrical, etc., a control system, a display, etc.

Actuator 14 may be coupled to an upper (proximal) portion of handle 12, and may be configured to control articulation of at least a portion of flexible shaft 15, and/or an articulation joint at a distal end of flexible shaft 15, in multiple directions. Actuator 14 may be, for example, one or more rotatable knobs that each rotates about its axis to push/pull actuating elements, e.g., steering wires (not shown). The actuating elements, such as cables or wires suitable for medical procedures (e.g., medical grade plastic or metal), extend distally from a proximal end of device 10 and connect to a distal portion of flexible shaft 15 to control movement thereof. Alternatively, or additionally, a user may operate actuating elements independently of handle 12, for example, via a separate control device. Distal ends of actuating elements may extend through flexible shaft 15 and terminate at an articulation joint and/or a distal cap 150 of flexible shaft 15. For example, one or more actuating elements may be connected to an articulation joint, and actuation of actuating elements may move the articulation joint and/or the distal end of flexible shaft 15 in multiple directions.

Handle 12 may further include one or more additional controls 17, e.g., a button. Although not shown, controls 17 may additionally or alternatively include one or more levers, rotational features, etc., which may be configured to actuate the expansion and/or contraction of the expandable features of distal cap 150, which are discussed in further detail below. For example, controls 17 may be actuated via depressing a button (as shown in FIG. 1) to expand the expandable feature from a retracted state and also retract the expandable feature from an expanded state. In other embodiments in which controls 17 includes a lever and/or a rotational feature, the lever and/or the rotational feature may be turned in a first direction, to expand the expandable feature from a retracted state, and turned in a section direction to retract the expandable feature from an expanded state. Controls 17 may engage the expandable features of distal cap 150 via any additional actuating elements, e.g., push/pull actuating elements, rotational elements, etc., extending between controls 17 and distal cap 150. The placement of controls 17 along handle 12, and in relation to actuator 14, is not particularly limited, and may be ergonomically placed. For example, controls 17 may be placed along a proximal portion of handle 12 configured for manipulation by an operator/user/physician's finger, e.g., index finger.

Handle 12 may also include one or more ports 13, 18 for introducing and/or removing tools, fluids, or other materials from the patient. For example, port 13 may be used to introduce an accessory device (not shown), which may be any suitable tool or device for medical purposes. Port 13 may be in fluid communication with a working channel, e.g., a lumen (not shown) of shaft 15. Although not shown, port 13 may be a Y-port. Port 13 may receive any suitable accessory device (e.g., a snare, a net, a basket, a forceps, a grasper, scissors, a clip, a stapler, a needle, a knife, an electrode, a cautery loop, etc.), which may extend distally throughout shaft 15 and towards the distal end of device 10. Port 18 may be connected to umbilicus 11, for example, for introducing fluid, applying suction, and/or coupling wiring for electronic components.

In addition, one or more electrical cables (not shown) may extend from the proximal end of device 10 (e.g., from handle 12 and/or umbilicus 11), through flexible shaft 15, to distal cap 150. The one or more electrical cables may provide power and/or electrical signals to imaging, lighting, and/or other electrical devices at distal cap 150, and may carry imaging signals from one or more imaging devices at distal cap 150 proximally to be processed and/or displayed on a display. Handle 12 is not particularly limited and may be any suitable scope handle configured for handling by an operator, e.g., a physician.

Shaft 15 may include at least one lumen (not shown) for receiving any number of additional devices, e.g., scopes, tools, instruments, cables, or the like, delivering fluid, applying suction, etc. Shaft 15 extends between a proximal end coupled to handle 12 and a distal end coupled to distal cap 150. Shaft 15 is not particularly limited, and may be any suitable flexible shaft configured to traverse bodily lumens during a procedure.

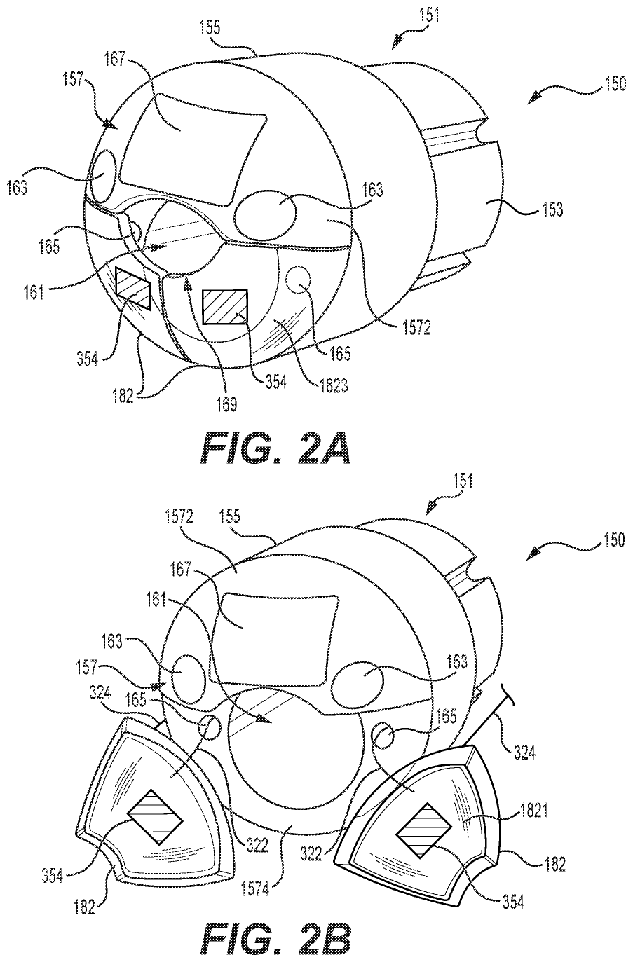

FIGS. 2A and 2B show an embodiment of distal cap 150 for a medical device, including, but not limited to, device 10. Distal cap 150 may be coupled to a distal end of shaft 15, and may include one or more components that are in communication with handle 12 and/or a control system or display, e.g., via umbilicus 11. Distal cap 150 includes a body 151, which may include a primary channel 161, one or more secondary channels 165 (e.g., a pair of secondary channels 165), one or more tertiary channels 163 (e.g., a pair of tertiary channels 163), and an imager socket 167. Primary channel 161 may be in communication with a central lumen, i.e., the working channel, of shaft 15, so that one or more instruments, tools, and accessories may traverse the working channel and primary channel 161, and extend distally from the distal opening of primary channel 161. The diameter of primary channel 161 may be approximately identical to the diameter of the working channel of shaft 15. For example, the diameter of primary channel 161 may be between approximately 0.5 mm and approximately 4.0 mm, and in other examples, may be between approximately 1.5 mm and approximately 2.0 mm. However, the diameter of primary channel 161 is not particularly limited, and may be of any suitable diameter that is able to accommodate one or more instruments, tools, and accessories that may be used in various medical procedures, e.g., endoscopy.

Secondary channels 165 may be in communication with respective lumens of shaft 15, and may be utilized for various purposes. For example, as shown in FIG. 2B (one of secondary channels 165 is covered by a wing 182 in FIG. 2A), secondary channels 165 (and the respective lumens of shaft 15) may each provide a passage for a strand of wire pair 322, as well as other additional cables, wires, etc., that may be in connection with lights 354. The diameters of secondary channels 165 are not particularly limited, and may be of any suitable diameters that accommodate one or more wires, cables, etc. The shape and/or size of secondary channels 165 is also not particularly limited, and may be, for example, cylindrical, as shown in FIGS. 2A and 2B. Each of secondary channels 165 may be adjacent to primary channel 161, and each of secondary channels 165 may be on diametrically opposite sides of primary channel 161, as shown in FIG. 2B.

Tertiary channels 163 may be in communication with respective irrigation or suction lumens of shaft 15. The diameters of tertiary channels 163 are not particularly limited, and may be any suitable diameters for irrigation or suction purposes. The shape and/or size of tertiary channels 163 is also not particularly limited, and may be, for example, cylindrical, as shown in FIGS. 2A and 2B. The position of tertiary channels 163 within body 151 is not particularly limited, and may be any suitable location for irrigation or suction purposes.

Imager socket 167 is not particularly limited, and may be of any shape and size to receive any suitable imaging device, e.g., a camera 351 (shown in FIG. 1). Imager socket 167 may be in communication with a respective lumen of shaft 15 configured to house wires, cables, fibers, etc. that may be coupled to an imager, e.g., camera 351. The position of imager socket 167 is not particularly limited, and may be, for example, positioned relative to primary channel 161, second channels 165, and tertiary channels 163, as shown in FIGS. 2A and 2B.

Body 151 and a distal face 157 of body 151 does not accommodate for light features. Rather, as shown in FIG. 2B and as further discussed below, lights 354 may be coupled to separate aspects of distal cap 150, e.g., wings 182. Given that lights 354 are coupled to wings 182, and are not present on distal face 157, wings 182 may provide the additional surface area on distal face 157 to accommodate for additional openings/channels, a larger imager, and/or a larger primary channel 161 (and working channel), relative to a shaft that also accommodates for light features on its distal face.

Body 151 may be defined by a proximal portion 153 and a distal portion 155. As shown in FIGS. 2A and 2B, proximal portion 153 may be of a smaller diameter than distal portion 155, but this disclosure is not limited thereto. Proximal portion 153 may be sheathed by a distal portion of the catheter/tube of shaft 15 so that a distal end of the catheter may be flush against a proximal surface of distal portion 155, for example, at a transition between proximal portion 153 and distal portion 155 of body 151. The manner in which proximal portion 153 is adhered to the catheter of shaft 15 is not particularly limited, and may include the use of adhesives, etc. Distal portion 155 may be of a diameter that is approximately the same as that of the outer diameter of the catheter of shaft 15, but this disclosure is not necessarily limited thereto.

Distal portion 155 includes distal face 157, which accommodates for various openings associated with each of the channels 161, 163, 165 and imager socket 167 discussed above. As shown in FIG. 2B, distal face 157 may be tiered so that a first portion 1572 of distal face 157 protrudes or extends distally relative to a second portion 1574 of distal face 157. Furthermore, first portion 1572 of distal face 157 may protrude in an at least partially-domed shape, thereby defining a partially rounded surface. However, distal face 157 is not necessarily limited to such a configuration, and in some examples, may be flat and/or uniform in degree of protrusion throughout the face. Second portion 1574 may be flat, but not necessarily limited to such a configuration.

Distal portion 155 may further include a pair of wings 182, for example, pivotably coupled to second portion 1574. Wings 182 may be semi-annular and convex-shaped, so that when wings 182 are in a closed, retracted position, as shown in FIG. 2A, wings 182 and first portion 1572 of distal face 157 define an opening 169, which may at least partially align with primary channel 161. The size of opening 169 is not particularly limited and may be of any suitable diameter that may allow for various tools, accessories, instruments, e.g., a guidewire, to extend therethrough, as well as through primary channel 161 and the working channel of shaft 15. Wings 182 may be of any suitable materials, e.g., medical grade plastic, and may also be transparent, but this disclosure is not limited thereto.

Each of wings 182 include an outer surface 1823 and an inner surface 1821. Light features, e.g., lights 354, may be coupled to inner surface 1821. The light feature is not particularly limited, and may be any suitable light including, but not limited to, LED, fiber optics, etc. The means by which lights 354 are coupled, e.g., adhesives, is not particularly limited, and may be via any suitable means. Moreover, lights 354 may be coupled so that illumination from lights 354 is emitted distally when wings 182 are in an open, expanded position (FIG. 2B), and light is emitted proximally when wings 182 are in a retracted position (FIG. 2A). Because wings 182 may be transparent, light that is emitted proximally may still be able to illuminate bodily lumens to some degree through transparent wings 182. Second portion 1574 may be at least partially reflective to allow for such illumination.

Wings 182 may be coupled to second portion 1574 via a hinge or a similarly-functioning feature that allows for wings 182 to transition between a retracted position, as shown in FIG. 2A, and an expanded position, as shown in FIG. 2B. In the expanded position, an entirety the distal opening of primary channel 161 may be exposed. In the retracted position, a portion of the distal opening of primary channel 161 may be exposed. The transition between the two configurations may be actuated via any suitable actuator. For example, controls 17 (shown in FIG. 1) may engage wings 182 via one or more suitable actuating elements, e.g., closing wires 322 and/or opening wires 324 (shown in FIG. 2B). Closing wires 322 and opening wires 324 are not particularly limited and may be, for example, nitinol wires. Each closing wire 322 may extend through one of secondary channels 165, and a distal end of each closing wire 322 may be coupled to inner surface 1821 of wings 182. Thus, a pulling force applied to closing wires 322 (i.e., pulling wires 322 proximally) may transition wings 182 from an expanded position to a retracted position. In some embodiments, each closing wire 322 may be two separate wire strands, each of which is coupled to separate controls 17, so that each wing 182 may be closed separately from the other. In other embodiments, each closing wire 322 may extend from a single drive wire strand coupled to a single actuator of controls 17 or may be coupled to a single actuation wire, so that both wings 182 may be closed simultaneously. Distal cap 150 may be configured so that its default state is a retracted position as shown in FIG. 2A. The retracted position may be maintained via one or more biasing elements. For example, controls 17 and/or another feature of handle 12 or distal cap 150 (e.g., a detent and/or a lever) may be configured to maintain each of closing wires 322 (or, in other embodiments, the single drive wire) in a tight, held position, unless actively engaged by a user. Thus, the retracted position may be a default configuration of distal cap 150.

Each opening wire 324 may extend along an exterior of distal cap 150 (or through side channels (not shown) of distal cap 150), and through the catheter of shaft 15). A distal end of each opening wire 324 may be coupled to outer surface 1823 of wings 182. Thus, a pulling force applied to opening wires 324 (i.e., pulling wires 324 proximally) may transition wings 182 to an expanded position. In some embodiments, each opening wire 324 may be two separate wire strands, each of which is coupled to separate controls 17, so that each wing 182 may be opened separately from the other. In other embodiments, each opening wire 324 may be from a single wire strand coupled to a single actuator of controls 17 or may be coupled to a single drive wire, so that both wings 182 may be opened simultaneously. The expanded position may also be maintained via one or more biasing elements. For example, controls 17 and/or another feature of handle 12 or distal cap 150 (e.g., a detent and/or a lever) may be configured to maintain each of opening wires 324 (or, in other embodiments, the single drive wire) in a tight, held position.

In other embodiments, distal cap 150 may only include a single pair of wires, e.g., wires 322. The wires may extend from a single drive wire strand (not shown) coupled to an actuator of controls 17. In such an embodiment, the default configuration of distal cap 150 may be the retracted position, and the single drive wire may be maintained in a default position by any suitable biasing/locking structure. From the default position, the single drive wire may be translated distally, for example, via a first force applied by the actuator, thereby extending wires 322 against wings 182 and transitioning distal cap 150 into the expanded position. The single drive wire may also be translated proximally towards its default position, via a second force applied by the actuator, thereby retracting wires 322 and wings 182 and transitioning distal cap 150 into a retracted position. Alternatively, the above-discussed biasing structure (or a different biasing structure), e.g., a spring, may return the single drive wire to its default position, thereby returning distal cap 150 into the retracted position.

Referring to FIGS. 1, 2A, and 2B, an example of how medical device 10 may be used is further discussed below. A user may deliver a distal end of a shaft of a mother scope, e.g., an endoscope into the body of a subject, e.g., via a natural orifice (such as a mouth or anus) and through a tortuous natural body lumen of the subject, such as an esophagus, stomach, colon, etc., towards an intended target site. A user may then deliver a guidewire through a channel of the shaft of the mother scope adjacent or aimed towards the intended target site. A user may then pass a proximal end of the guidewire through opening 169 of distal cap 150 in the retracted position, and deliver a distal end of shaft 15 of device 10, over the guidewire and through the channel of the shaft of the mother scope. Once the distal end of shaft 15 is delivered to the intended target site, via the guidewire, a user may actuate controls 17 to transition distal cap 150 from the retracted position (FIG. 2A) to the expanded position (FIG. 2B). A user may maintain distal cap 150 in the expanded position via a locking mechanism that may be actuated via controls 17. A user may then activate lights 354 via any suitable means, thereby emitting light distally. A user may then remove the guidewire from the working channel. A user may irrigate the targeted site via an actuator or control on handle 12 or a separate control device. A user may image the intended target site, via camera 351, which may be controlled via another actuator or control on handle 12 or a separate control device. A user may also introduce additional tools or instruments via the working channel of shaft 15. A user may also actuate controls 17 to transition distal cap 150 from the expanded position to the retracted position, before retracting the distal end of shaft 15 of device 10 from the body of the subject or before deflecting the shaft of the mother scope to image/treat the targeted site in a different position/orientation. Alternatively, distal cap 150 may be biased towards a retracted position, and a user may allow distal cap 150 to revert to its biased retracted position. A user may actuate controls 17, again, to expand wings 182 and transition distal cap 150 to an expanded position, for any reason.

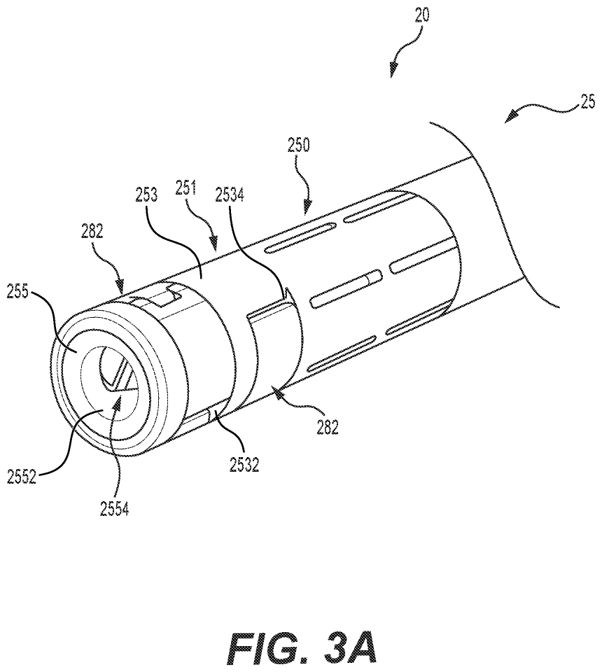

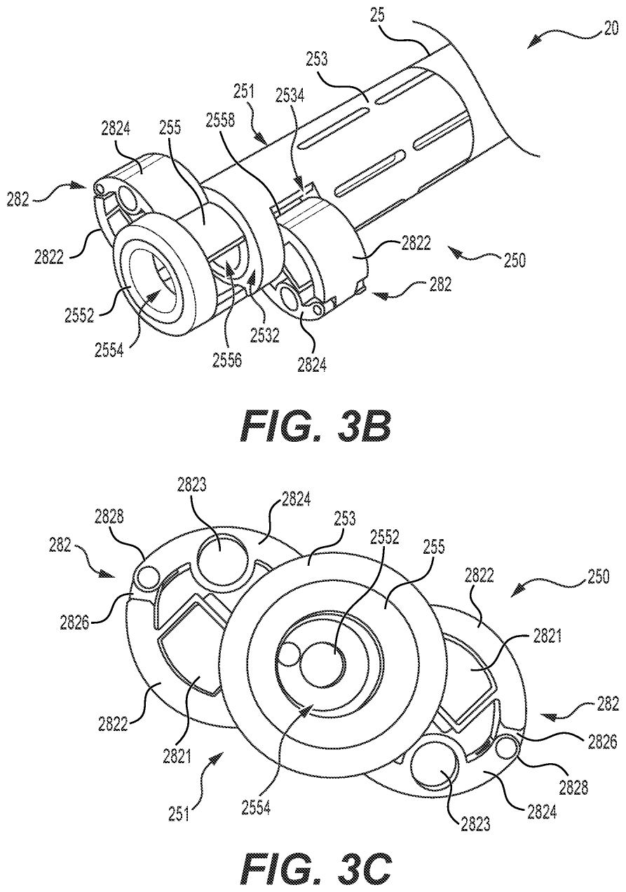

FIGS. 3A-3C illustrate another embodiment of a medical device 20 and distal cap 250 coupled to a distal portion of shaft 25, according to aspects of this disclosure. Like reference numerals refer to like parts. Distal cap 250 includes a body 251 comprising an outer cylinder 253, and outer cylinder 253 may sheath or otherwise enclose at least a portion of a rotatable inner cylinder 255. Outer cylinder 253 and inner cylinder 255 may thus form a two-cylinder overlay, and a pair of wings 282 coupled to body.

Outer cylinder 253 includes a lumen (not shown) extending between a proximal end and a distal portion of outer cylinder 253 (FIG. 3B). The lumen receives and at least partially sheaths inner cylinder 255. The size and/or shape of the lumen is not particularly limited, and may be of a diameter allowing for the rotation of inner cylinder 255 about a central longitudinal axis. The proximal end is not particularly limited and may be any suitable end configured to be attached to and/or flush against a distal end of the catheter/tube of shaft 25 of device 20, and to also receive at least a portion of inner cylinder 255. The distal end is also not particularly limited, and may include a distal opening of a diameter that is approximately the same as that of an outer diameter of inner cylinder 255. The dimensions of outer cylinder 253 is not particularly limited. In some examples, outer cylinder 253 may be of a diameter that is approximately the same as that of the outer diameter of shaft 25. Moreover, the material of outer cylinder 253 is not particularly limited and may be of any suitable flexible, biocompatible plastic.

Outer cylinder 253 includes a first slot 2532 and a second slot 2534. As shown in FIGS. 3A and 3B, slots 2532, 2534 extend around a portion of the circumference of outer cylinder 253. The dimensions of slots 2532, 2534 are not particularly limited, and may be of any suitable width that may accommodate expandable wings 282. Moreover, slots 2532, 2534 may be of any suitable depth so that an outer diameter of body 251 is consistent, or approximately consistent, throughout its length when wings 282 are retracted and distal cap 250 is in a retracted position. Slots 2532, 2534 are spaced apart from one another, along the length of outer cylinder 253. Slots 2532, 2534 may also be offset from one another. For example, slot 2532 may extend around a first portion of the circumference of outer cylinder 253 and slot 2534 may extend around a second portion of the circumference of outer cylinder 253. Although the first portion and the second portion may overlap over a circumference of outer cylinder 253, the first portion and the second portion do not span the same circumferential portions of outer cylinder 253. Such offset may allow for wings 282 to expand in different radial directions.

As shown in FIGS. 3A-3C, inner cylinder 255 includes a lumen extending between a proximal end and a distal end. The diameter of the lumen of inner cylinder 255 is not particularly limited, and may be of a diameter that is able to accommodate instruments, tools, and accessories that may be used in various medical procedures, e.g., endoscopy. The proximal end is not particularly limited and may be any suitable end configured to be coupled to or engage a distal end of the catheter/tube of shaft 25 of device 20. The distal end is also not particularly limited, and may include a distal opening 2552. Thus, distal opening 2552 of inner cylinder 255 and the lumen of the inner cylinder 255 may define a primary channel 2554 extending between a proximal end and a distal end of body 251 of distal cap 250. Primary channel 2554 may be in communication with the working channel of shaft 25 so that instruments, tools, and accessories may traverse the working channel of shaft 25 and primary channel 2554 of distal cap 250, and extend distally from distal opening 2552. The diameter of primary channel 2554 is not particularly limited, and may be of any suitable diameter that is able to accommodate instruments, tools, and accessories that may be used in various medical procedures, e.g., endoscopy. The alignment between primary channel 2554 and the working channel of shaft 25 may be maintained while wings 282 are retracted and distal cap 250 is in the retracted position. Furthermore, wings 282 do not interfere with the distal delivery of instruments, tools, and/or accessories when expanded and distal cap 250 is in the expanded position. The diameter of primary channel 2554 may be approximately identical to the diameter of the working channel of shaft 25. For example, primary channel 2554 may include a diameter between approximately 0.5 mm and approximately 4.0 mm, and in other examples, may be between approximately 1.5 mm and approximately 2.0 mm. However, the diameter of primary channel 2554 is not particularly limited, and may be of any suitable diameter that is able to accommodate one or more instruments, tools, and accessories that may be used in various medical procedures, e.g., endoscopy.

Inner cylinder 255 further includes actuating elements (not shown), e.g., rotational wires, gears, knobs, etc., that are configured to rotate inner cylinder 255 when actuated via actuators, e.g., controls 17, present on handle 12, while outer cylinder 253 remains stationary. Thus, said actuating elements may engage and extend between both inner cylinder 255 and controls 17 via any suitable means, for example, to extend and/or retract one or more of wings 282 relative to body 251 of distal cap 250. Controls 17 and/or another feature of handle 12 (e.g., a detent and rotational knob) may be configured to lock/maintain distal cap 250 in an expanded position or retracted position.

Inner cylinder 255 includes a first slot 2556 and a second slot 2558. Slots 2556, 2558 extend around a portion of the circumference of inner cylinder 255. The dimensions of slots 2556, 2558 are not particularly limited, and may be of any suitable width that may accommodate expandable wings 282. Thus, slots 2532, 2534 of outer cylinder 253 and slots 2556 and 2558 of inner cylinder 255 may be overlaid. Slots 2556, 2558 are also spaced apart from one another, along the length of inner cylinder 255, and slots 2556, 2558 are also offset from one another to be in accordance with offset slots 2532, 2534 of outer cylinder 253. For example, slot 2556 may extend around a first portion of the circumference of inner cylinder 255 and slot 2558 may extend around a second portion of the circumference of inner cylinder 255. Although the first portion and the second portion may overlap over a circumference of inner cylinder 255, the first portion and the second portion do not span the same circumferential portions of inner cylinder 255. Inner cylinder 255 is not particularly limited and may be formed of a flexible but torqueable material, e.g., a nitinol tube or hollow drive cable.

Wings 282 are respectively fitted within slots 2532, 2556 and slots 2534, 2558, and wings 282 are configured to expand and retract from the aforementioned slots as discussed in further detail below. While two wings 282 are shown in FIGS. 3A-3C, the number of wings is not limited thereto, and may range from one wing to more than two wings.

Each of wings 282 include a first portion 2822 and a second portion 2824. First portion 2822 may be configured to fit within one of slots 2556, 2558 of inner cylinder 255 and the corresponding slot of outer cylinder 253, when wings 282 are in a retracted position. In some examples, first portion 2822 may be of a partial cylindrical shape, but this disclosure is not limited thereto. First portion 2822 may include a first socket 2821. First socket 2821 is not particularly limited, and may be of any shape and size to receive and retain any suitable imaging device, e.g., camera 351 (FIG. 1). Second portion 2824 may also be configured to fit within one of slots 2556, 2558 of inner cylinder 255 and the corresponding slot of outer cylinder 253, when wings 282 are in the retracted position. In some examples, second portion 2824 may be of a partial cylindrical shape, but this disclosure is not limited thereto. Second portion 2824 may include a second socket 2823. Second socket 2823 is not particularly limited, and may be of any shape and size to receive and hold any suitable light feature, e.g., one or more of lights 354 (shown in FIG. 2B). Cables extending from the camera and light features may extend through outer cylinder 253, which may further include additional lumen(s) configured to receive and sheath such cables.

Each of first portion 2822 and second portion 2824 includes a first end 2826, 2828 and a second end (not shown). First end 2826 of first portion 2822 and first end 2828 of second portion 2824 may each have hinge forming features so that first end 2826 and first end 2828 may engage one another via a hinged relationship. A second end of first portion 2822 may also engage an inner surface of outer cylinder 253 defining slots 2532, 2534, so that first portion 2822 may hinge relative to outer cylinder 253. A second end of second portion 2824 may also engage an inner surface of inner cylinder 255 defining slots 2556, 2558, so that second portion 2824 may hinge relative to inner cylinder 255. Thus, in view of such configuration, the rotation of inner cylinder 255, via an actuator, e.g., controls 17 of handle 12, may control the expanding and retracting of wings 282. As inner cylinder 255 rotates in a first direction, e.g., clockwise when viewing from the proximal end of cap 250, first portion 2822 and second portion 2824 may hinge towards one another and hinge away from inner cylinder 255, thereby transitioning into an open, expanded position as shown in FIG. 3B. Alternatively, as inner cylinder 255 rotates in a second direction, e.g., counter-clockwise when viewing from the proximal end of cap 250, first portion 2822 and second portion 2824 hinge away from one another and towards inner cylinder 255, thereby transitioning into a closed, retracted configuration as shown in FIG. 3A. In other embodiments, the first direction may be counter-clockwise when viewing from the proximal end of cap 250 and the second direction may be clockwise when viewing from the proximal end of cap 250. Distal cap 250 may be in a closed, retracted configuration by default. Thus, device 20 may be used in a similar as device 10, as discussed above.

Thus, lights and imaging devices may be coupled to separate aspects of distal cap 250, e.g., wings 282, as opposed to a distal face of distal cap 250, thereby providing additional surface area on the distal face to accommodate for a larger primary channel 2554 (and working channel) and/or additional channels or features (not shown), relative to a device that also accommodates for lights and imaging devices on its distal face. Furthermore, given that lights and imaging devices may be coupled to wings 282, said lights and/or imaging devices may also be larger. This may provide greater illumination and/or imaging relative to a device that also accommodates for lights and imaging devices on its distal face.

It will be apparent to those skilled in the art that various modifications and variations can be made to the disclosed device without departing from the scope of the disclosure. Other embodiments of the disclosure will be apparent to those skilled in the art from consideration of the specification and practice of the invention disclosed herein. It is intended that the specification and examples be considered as exemplary only, with a true scope and spirit of the invention being indicated by the following claims.

What is claimed is:

1. A medical device, comprising:
a handle including an actuator;
a shaft extending distally from the handle, wherein the shaft includes a lumen; and
a distal cap, wherein the distal cap includes:
a body including a first channel, wherein the first channel is in communication with a distal portion of the lumen,
a distal face including an opening, wherein the opening is a distal opening of the first channel, and
an expandable portion coupled to the body, wherein the expandable portion is configured to expand radially outwards from the body and to retract radially inwards towards the body from an expanded position, and wherein the expandable portion includes at least one light.

2. The medical device of claim 1, wherein the actuator is in engagement with the expandable portion so that the actuator is configured to control expansion and retraction of the expandable portion.

3. The medical device of claim 1, wherein the expandable portion is biased towards a retracted position.

4. The medical device of claim 3, wherein in the retracted position, an outer diameter of the distal cap is approximately the same as an outer diameter of a remaining portion of the shaft.

5. The medical device of claim 1, wherein the distal face does not include any lights.

6. The medical device of claim 1, wherein a diameter of the opening is about 1.5 mm to 2.0 mm.

7. The medical device of claim 1, wherein the expandable portion is coupled to the body via a hinge.

8. The medical device of claim 7, wherein the expandable portion is coupled to a distal end of the body so that the expandable portion covers at least a portion of the opening in a retracted position and exposes an entirety of the opening in the expanded position.

9. The medical device of claim 8, wherein the expandable portion is semi-annular so that in the retracted position a portion of the opening remains exposed.

10. The medical device of claim 8, wherein the expandable portion is transparent.

11. The medical device of claim 8, wherein the actuator and the expandable portion engage one another via an opening wire and a closing wire, wherein a force applied to the opening wire is configured to expand the expandable portion, and wherein a force applied to the closing wire is configured to retract the expandable portion.

12. The medical device of claim 7, wherein the body further includes an outer cylinder and an inner cylinder, wherein the outer cylinder encloses at least a portion of the inner cylinder and the inner cylinder is rotatable about a central longitudinal axis of the body.

13. The medical device of claim 12, wherein the outer cylinder includes a first slot and the inner cylinder includes a second slot, wherein the first slot and the second slot are overlaid and the first slot and the second slot are configured to receive the expandable portion in a retracted position.

14. The medical device of claim 13, wherein the expandable portion is coupled to the outer cylinder via a hinge and the inner cylinder via a hinge, and a rotational force applied to the inner cylinder in a first direction is configured to expand the expandable portion.

15. The medical device of claim 14, wherein a rotational force applied to the inner cylinder in a second direction is configured to retract the expandable portion into the first slot and the second slot.

16. A medical device end cap, comprising:
a body including a channel,
a distal face including an opening, wherein the opening is a distal opening of the channel,
a first wing coupled to the body, and
a second wing coupled to the body, wherein each of the first wing and the second wing is configured to expand radially outwards from the body to an expanded position and to retract radially inwards towards the body from the expanded position, and wherein each of the first wing and the second wing includes at least one light.

17. The medical device end cap of claim 16, wherein the first wing is configured to expand radially outwards in a first radial direction and the second wing is configured to expand radially outwards in a second radial direction, wherein the first radial direction and the second radial direction are different.

18. The medical device end cap of claim 16, wherein the distal face does not include any lights.

19. The medical device end cap of claim 16, wherein one or more of the first wing and the second wing further includes an imaging device.

20. A method of using a medical device, the medical device including a handle including an actuator, a shaft, and a distal cap including an expandable portion controllable by the actuator, wherein the expandable portion includes at least one light, the method comprising:
delivering a distal end of the shaft and the distal cap into a bodily lumen;
positioning the distal end of the shaft and the distal cap adjacent to a target site;
actuating the actuator to control the expandable portion of the distal cap to expand the expandable portion of the distal cap; and
activating the at least one light.
